SEQUENCE LISTING (4 sequences)

SEQ ID NO: 1 - DNA, 34 bp, synthetic construct
  source 1..34: mol_type = other DNA
gatgtgcgtcttgatcgagacctgcgacaggaag

SEQ ID NO: 2 - DNA, 33 bp, synthetic construct
  source 1..33: mol_type = other DNA
  misc_feature 19: note = RNA
  misc_feature 1..18: note = DNA
  misc_feature 20..33: note = DNA
tttttactcttcctagctatggttcgatcaaga

SEQ ID NO: 3 - DNA, 34 bp, synthetic construct; identical to SEQ ID NO: 1
  source 1..34: mol_type = other DNA
  misc_feature 1: note = acrydite modification

SEQ ID NO: 4 - DNA, 33 bp, synthetic construct; identical to SEQ ID NO: 2
  source 1..33: mol_type = other DNA
  misc_feature 1: note = acrydite modification
  misc_feature 1..18: note = DNA
  misc_feature 19: note = RNA
  misc_feature 20..33: note = DNA

(19) United States
(12) Patent Application Publication
(10) Pub. No.: US 2026/0243764 A1
(43) Pub. Date: Aug. 20, 2026

(54) BIOSENSORS AND RELATED METHODS

(71) Applicant: McMaster University, Hamilton (CA)

(72) Inventors: Tohid Didar, Dundas (CA); Carlos Filipe, Ancaster (CA); Hannah Mann, Hamilton (CA); Shadman Khan, Toronto (CA); Akansha Prasad, Markham (CA)

(21) Appl. No.: 19/350,730

(22) Filed: Oct. 6, 2025

Related U.S. Application Data
(60) Provisional application No. 63/703,447, filed on Oct. 4, 2024.

(51) Int. Cl. G01N 33/569; C12N 7/00; G01N 21/78

(52) U.S. Cl. CPC G01N 33/56916; C12N 7/00; G01N 21/78; C12N 2795/00031; G01N 2333/245

(57) ABSTRACT

Described herein is a biosensor for detecting the presence of a target molecule in a sample. The biosensor comprises: a polymeric hydrogel; a colorimetric indicator entrapped in the polymeric hydrogel; and an oligonucleotide that crosslinks the polymeric hydrogel, wherein the oligonucleotide is separated in the presence of the target molecule, thereby releasing the colorimetric indicator from the hydrogel.

### CROSS-REFERENCE TO RELATED APPLICATION

This application claims priority to and the benefit of U.S. Provisional Patent Application No. 63/703,447, filed Oct. 4, 2024, the content of which is hereby incorporated herein by reference in its entirety.

### STATEMENT REGARDING ELECTRONIC FILING OF A SEQUENCE LISTING

A Sequence Listing XML file, submitted under 37 C.F.R. §§ 1.831-835, entitled “Sequence Listing March-2026 16112-70.xml”, 6,788 bytes in size, and created on Mar. 27, 2026. The Sequence Listing is incorporated herein by reference in its entirety into the specification for its disclosures.

### FIELD

The present disclosure relates to molecule detection, and in particular, to a biosensor and methods of making and uses thereof.

### BACKGROUND

DNAzymes are synthetic DNA molecules with catalytic activity that enhance food and water safety by providing sensitive, cost-effective, and portable detection methods for contaminants like pathogens, heavy metals, and pesticides. Their peroxidase-like activity or RNA-cleaving activity enable rapid, on-site detection via colorimetric, fluorescent, or electrochemical signals, overcoming the limitations of traditional methods. Innovations like DNAzyme-printed food packaging and nanoparticle-based systems further improve real-time monitoring and on-site testing. Despite this, there is a need for low-cost and fast methods of detecting molecules, such as from bacteria, without the need for complex or specialized equipment.

The background herein is included solely to explain the context of the disclosure. This is not to be taken as an admission that any of the material referred to was published, known, or part of the common general knowledge as of the priority date.

### SUMMARY

In accordance with an aspect, there is provided a biosensor for detecting the presence of a target molecule in a sample, the biosensor comprising: a polymeric hydrogel; a colorimetric indicator entrapped in the polymeric hydrogel; and an oligonucleotide that crosslinks the polymeric hydrogel, wherein the oligonucleotide is separated in the presence of the target molecule, thereby releasing the colorimetric indicator from the hydrogel.

In an aspect, the target molecule is a protein from a bacterial target.

In an aspect, the bacterial target comprises E. coli.

In an aspect, the biosensor further comprising a lytic bacteriophage that is capable of infecting and lysing the bacterial target, thereby increasing the concentration of the protein in the sample.

In an aspect, the bacteriophage comprises Bacteriophage T7.

In an aspect, the colorimetric indicator comprises gold nanoparticles.

In an aspect, the colorimetric indicator is bulked with a BSA coating.

In an aspect, the polymeric hydrogel comprises acrylamide.

In an aspect, the oligonucleotide is acrydite-modified.

In an aspect, the oligonucleotide comprises a substrate strand hybridized to a DNAzyme strand, wherein the DNAzyme strand cleaves the substrate strand in the presence of the protein specific to the target.

In an aspect, the DNAzyme strand comprises the sequence GA TGT GCG TCT TGA TCG AGA CCT GCG ACA GGA AG (SEQ ID NO: 1).

In an aspect, the substrate strand comprises the sequence TT TTT ACT CTT CCT AGC TrAT GGT TCG ATC AAG A (SEQ ID NO: 2).

In an aspect, the biosensor has a sensitivity of greater than about 101 CFU mL−1.

In an aspect, the biosensor produces a visible result in less than about 18 hours.

In an aspect, no equipment is needed for analysis of the test result.

In an aspect, the biosensor is stable for at least two weeks at temperatures between about −20° C. and about 20° C.

In an aspect, the sample is a liquid sample from water, a bodily fluid, a food source, and/or a surface.

In accordance with an aspect, there is provided a method of detecting a target molecule in sample, the method comprising applying the sample to the biosensor described herein.

In an aspect, the method further comprises use of an AI network to assess the colorimetric result.

In accordance with an aspect, there is provided a method of amplifying DNAzyme signalling in a biosensor that detects a bacteria-specific protein from whole bacteria, the method comprising applying to the sensor a lytic bacteriophage that infects and lyses the bacteria.

In accordance with an aspect, there is provided a bacterial sensing platform comprising:
a. One or more probes that detect intracellular bacterial targets
b. One or more bacteriophages
   Wherein bacteriophage-mediated targeted bacterial cell lysis results in increased concentration of the probe's target, further amplifying probe signaling and improving platform sensitivity.

In an embodiment, the probe comprises functional oligonucleotide probes.

In an embodiment, the probe comprises DNAzymes.

In an embodiment, the bacterial sensing platform further comprises a hydrogel.

In an embodiment, the bacterial sensing platform further comprises a colorimetric indicator.

In an embodiment, the hydrogel comprises a polyacrylamide hydrogel.

In an embodiment, the colorimetric indicator comprises gold nanoparticles (AuNPs).

In an embodiment, the hydrogel is crosslinked with DNAzyme/substrate complex and embedded with gold nanoparticles (AuNPs), wherein colorimetric transduction occurs after DNAzyme cleavage of its substrate in response to detection of its bacterial target, breaking down the hydrogel and releasing the AuNPs.

In an embodiment, the substrate and DNAzyme oligonucleotide sequences are modified at the 5′ end with an acrydite group, allowing them each to be co-polymerized with acrylamide in free radical polymerization (FRP).

In an embodiment, the FRP comprises concentrations of about 10%-12.5% acrylamide monomer, and about 0.3-0.5 mM acrydite-modified DNA.

In an embodiment, the hydrogel is polymerized with concentrations comprising 10% acrylamide monomer and 0.4 mM acrydite-modified DNA.

In an embodiment, the DNAzyme is in a trans conformation.

In an embodiment, the colorimetric indicator comprises a visible colour change as the red colour from the AuNPs dissipates, which can be interpreted with the naked eye and/or with an artificial intelligence (AI) network.

In an embodiment, the visible colour change occurs at initial bacterial target concentrations of at least 101 CFU/mL.

In an embodiment, the bacterial target comprises a target for which a DNAzyme exists, including E. coli, Legionella, Salmonella.

In an embodiment, the bacterial target comprises E. coli.

In an embodiment, the hydrogel is stored in tubes, microwells or patches.

In an embodiment, the hydrogel can be stored for at least two weeks and at temperatures between −20° C. and 20° C.

In an embodiment, bacteria can be detected in samples in liquid form, including environmental samples such as water and clinical samples such as urine.

In a further embodiment, a method to prepare the bacterial sensing platform is provided, the method comprising:
a) Preparing two separate solutions, each containing an acrylamide monomer solution and one of the two acrydite modified oligonucleotide sequences, and heating to about 45° C.
b) Inducing free radical polymerization in both solutions with the addition of Ammonium persulfate (APS) as the initiator, and N,N,N′N′-Tetramethyl ethylenediamine (TEMED) catalyst.
c) Placing the solutions in a vacuum dessicator.
d) Adding water and AuNPs after polymerization
e) Mixing the polymer solutions grafted to each DNA oligo on a heating block at 95° C.
f) Cooling the hydrogels room temperature

In an embodiment, concentrations comprising 0.11-0.14% Ammonium persulfate (APS) as the initiator, and 0.07-0.21% N,N,N′,N′-Tetramethyl ethylenediamine (TEMED) catalyst are used.

In an embodiment, concentrations comprising 0.14% Ammonium persulfate (APS) as the initiator, 0.14% N,N,N′,N′-Tetramethyl ethylenediamine (TEMED) catalyst are used.

In a further embodiment, a method for detecting a bacterial target in a sample is provided, the method comprising:
a) Adding a buffer to the bacterial sensing platform of claims 1-19 to soak into the gel.
b) Adding yeast extract and the sample being tested
c) Incubating while shaking at 37-40° C. for about 6 hrs
d) Adding bacteriophage and incubating for about 12 hrs

In an embodiment, the sample comprises a liquid sample.

In an embodiment, the liquid sample comprises environmental samples such as water and clinical samples such as urine.

In an embodiment, the buffer comprises MgCl2.

In a further embodiment, a kit comprising the bacterial sensing platform and instructions for use thereof is provided.

Other features and advantages of the present disclosure will become apparent from the following detailed description. It should be understood, however, that the detailed description and the specific examples, while indicating embodiments of the disclosure, are given by way of illustration only and the scope of the claims should not be limited by these embodiments, but should be given the broadest interpretation consistent with the description as a whole.

### DRAWINGS

Certain embodiments of the disclosure will now be described in greater detail with reference to the attached drawings in which:

FIG. 1 shows Schematics of colorimetric E. coli detecting platform in exemplary embodiments of the application. a) Chemical structure of the polymeric matrix showing an acrydite DNAzyme/substrate crosslink. b) Macro and microscale schematics of the hydrogel matrix performance under contaminated or uncontaminated conditions. c) Intended functionality of the sensor under contaminated sample conditions.

FIG. 2 shows DNAzyme construct, shown without acrydite modifications in an exemplary embodiment of the application (SEQ ID NO: 1 and 2).

FIG. 3 shows 2D chemical structure of the polymeric matrix showing an acrydite DNAzyme/substrate crosslink breaking down at its cleavage site in an exemplary embodiment of the application (SEQ ID NO: 3 and 4).

FIG. 4 shows a schematic demonstrating the complete process of using the gel sensor platform in an exemplary embodiment of the application.

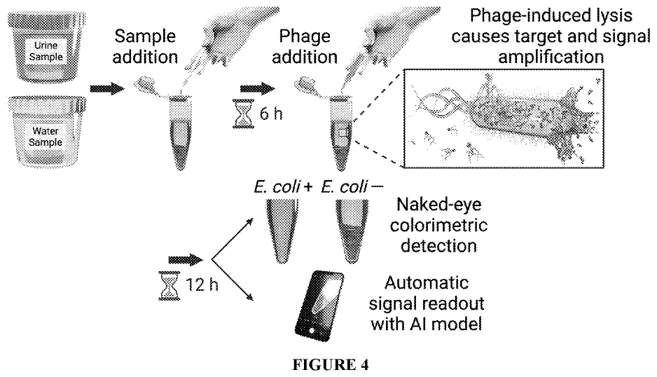

FIG. 5 shows the characterization and optimization of hydrogel detection system in exemplary embodiments of the application. a) Chemical diagram showing how an acrydite modified oligonucleotide fragment is added to a polyacrylamide chain during FRP. b)-e) Sensitivity testing of gels polymerized using different concentrations of APS, TEMED, acrylamide monomer, and acrydite modified DNA, respectively. Sensitivity was evaluated at 105 and 107 CFU mL−1 of E. coli. Sample size n=3 for each test category. f) Optical images of the colorimetric detection platform, showing a negative and positive result. Scale bars represent 5 mm. g) SEM images of freeze-dried hydrogel structure at 100× and 1000× magnification. h) Rheology result of a strain sweep performed at a fixed frequency of 1 Hz at 25° C. All asterisks represent significant differences at corresponding significance levels, and error bars represent standard error of the mean.

FIG. 6 shows chemical structures of reagents involved in the FRP reaction in exemplary embodiments of the application.

FIG. 7 shows SEM Images of hydrogel at different magnifications in exemplary embodiments of the application.

FIG. 8 shows rheology time sweep comparison of each sequence and the formed hydrogel in exemplary embodiments of the application. ACD refers to the polymer with the DNAzyme oligo, while ACR refers to the polymer with the substrate oligo.

FIG. 9 shows rheology strain sweep comparison of each sequence and the formed hydrogel in exemplary embodiments of the application. ACD refers to the polymer with the DNAzyme oligo, while ACR refers to the polymer with the substrate oligo.

FIG. 10 shows bacteriophage and machine learning integration, applied towards environmental sample testing in exemplary embodiments of the application. a) Optical images of colorimetric detection of E. coli after an 18 hour incubation at 40° C., with and without the addition of T7 phage after 6 hours. Numbers represent CFU mL−1. b)-c) Quantified results for the experiments pictured in part A. Each dataset's brightness values have been normalized to their respective controls. Panel B shows results without phage, panel C shows results with phage introduced after 6 hours. Sample size n=7 for each concentration. d) Schematic of the proposed mechanism of phage increasing DNAzyme cleavage. T7 bacteriophage attaching to E. coli, injecting its DNA, and inducing bacterial cell lysis, resulting in increased amounts of bacterial proteins being released into the extracellular environment. e) Electrophoresis gel results quantifying the percentage of DNAzyme that gets cleaved by different concentrations of E. coli, with and without phage. Measurements taken after 24 hours. f) Detection performance of the platform for 101 and 102 CFU mL−1 E. coli lake water samples. Sample size n=6 for each concentration. g) Detection performance of the platform for 103 CFU mL−1 E. coli cistern water samples. n=5 for each concentration. h) Validation accuracy and loss plots from the CNN trained on the colorimetric gel images. i) Schematic of AI analysis of gel images using a CNN. j) Confusion matrix of the AI model output when assessed with a testing dataset of 67 images. All asterisks represent significant differences at corresponding significance levels, and error bars represent standard error of the mean.

FIG. 11 shows sample optical images of hydrogel at several time points in exemplary embodiments of the application.

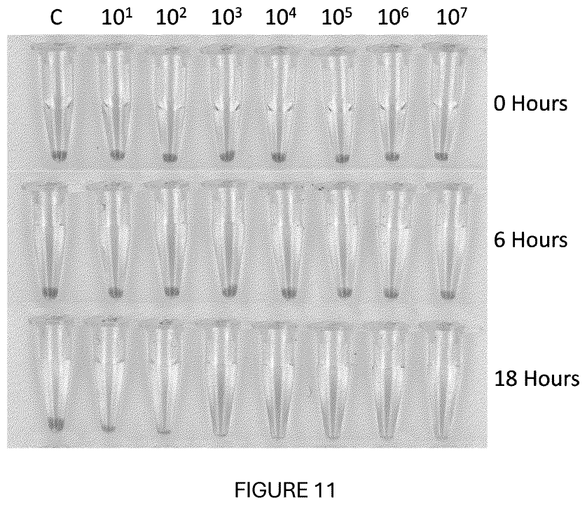

FIG. 12 shows detection performance of the platform for 103 CFU mL−1 E. coli K12 with lake water samples in exemplary embodiments of the application.

FIG. 13 shows training accuracy/loss plots from the CNN AI model in exemplary embodiments of the application.

FIG. 14 shows sensor specificity, stability, and performance against clinical urine samples in exemplary embodiments of the application. a) Detection specificity of the platform when tested with various other species of bacteria at concentrations of 103 and 106 CFU mL−1. Bacterial species are as follows: AL—Acinetobacter lwoffii, BS—Bacillus subtilis, LM—Listeria monocytogenes, PA—Pseudomonas aeruginosa, and MRSA—methicillin-resistant Staphylococcus aureus. Sample size n=6 for each concentration. b) Optical images showing naked-eye detection of E. coli 0157:H7 at concentrations of 103 and 106 CFU mL−1. Image taken after 18 hours at 40° C. c) Detection stability after storage for two weeks under different temperature conditions. n=7 at each condition. d) Detection performance of the platform for 106 CFU mL−1 E. coli with water samples of differing pH values. n=4 at each condition. e) Schematic demonstrating the potential use of this sensing platform for at-home UTI detection. f) Detection performance of the platform on healthy urine samples spiked with 105 CFU mL−1 of E. coli. n=4 at each condition. g) Detection performance of the platform on both healthy urine samples and E. coli contaminated urine samples from patients with UTIs. n=5 healthy samples and 7 infected samples. h) Optical images showing naked-eye detection of E. coli contaminated clinical urine samples. All asterisks represent significant differences at corresponding significance levels, datasets have brightness values normalized to their respective controls, and error bars represent standard error of the mean.

FIG. 15 shows box and whisker plot of the pH of clinical urine samples used, categorized by UTI status in exemplary embodiments of the application.

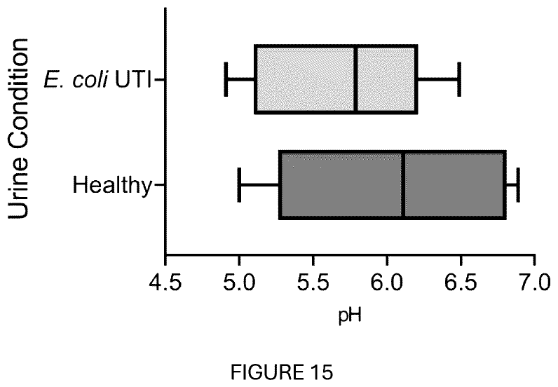

FIG. 16 shows sensor performance and workflow with leafy greens. a) Optical images of E. coli detection for lettuce. b) Detection performance for lettuce at 105 CFU mL−1 E. coli. Sample size n=4 at each concentration. c) Optical images of E. coli detection for salad mix. d) Detection performance for salad mix at 103 and 105 CFU mL−1 E. coli. Sample size n=4 at each concentration. e) Schematic of the process to extract and test a liquid sample from contaminated leafy greens. f) E. coli colony counts extracted from the spiked samples of leafy greens after stomaching, plotted on a log 10 scale. Each bar is a separate stomaching test, with n=3 technical replicates for each. g) Optical images of E. coli detection in the stomached leafy greens samples. h) Detection performance for stomached leafy greens at initial spiked concentrations of 105 and 107 CFU g−1 E. coli. Sample size n=4 at each concentration. Brightness values on each graph have been normalized to the mean of their respective controls. All asterisks represent significant differences at corresponding significance levels, and error bars represent standard error of the mean. Schematics created using Biorender.

FIG. 17 shows bacterial detection and sensor performance with various food products. a) Categories of foods that can be contaminated with pathogenic E. coli, and their known avenues of contamination. b) Optical images of E. coli detection in milk. c) Detection performance in milk at 105 CFU mL−1 E. coli. Sample size n=4 at each concentration. d) Optical images of E. coli detection in RTE chicken purge. e) Detection performance in RTE chicken purge at 105 CFU mL−1 E. coli. Sample size n=4 at each concentration. f) Optical images of E. coli detection in RTE eggs. g) Detection performance in RTE eggs at 105 CFU mL−1 E. coli. Sample size n=5 at each concentration. h) Optical images of E. coli detection in carrots. i) Detection performance in carrots at 105 CFU mL−1 E. coli. Sample size n=4 at each concentration. All bacterial concentrations are given as CFU mL−1. Brightness values on each graph have been normalized to the mean of their respective controls. All asterisks represent significant differences at corresponding significance levels, and error bars represent standard error of the mean. Schematics created using Biorender.

FIG. 18 shows microscale and macroscale schematics of the sensing platform. a) Microscale functionality of the sensing platform. Phage infection of E. coli bacteria and the resulting cell lysis increases the release of target protein. This target protein triggers DNAzyme cleavage and breakdown of the polyacrylamide hydrogel crosslinking. b) Macroscale depiction of the hydrogel sensing platform. An intact gel is visible in the absence of E. coli, while a contaminated sample results in a visibly degraded gel. Schematics created using Biorender.

### DETAILED DESCRIPTION

### I. Definitions

Unless otherwise indicated, the definitions and embodiments described in this and other sections are intended to be applicable to all embodiments and aspects of the present disclosure herein described for which they are suitable as would be understood by a person skilled in the art. It is also to be understood that the terminology used herein is for the purpose of describing particular aspects only and is not intended to be limiting.

In understanding the scope of the present disclosure, the term “comprising” and its derivatives, as used herein, are intended to be open ended terms that specify the presence of the stated features, elements, components, groups, integers, and/or steps, but do not exclude the presence of other unstated features, elements, components, groups, integers and/or steps. The foregoing also applies to words having similar meanings such as the terms, “including”, “having” and their derivatives. The term “consisting” and its derivatives, as used herein, are intended to be closed terms that specify the presence of the stated features, elements, components, groups, integers, and/or steps, but exclude the presence of other unstated features, elements, components, groups, integers and/or steps. The term “consisting essentially of”, as used herein, is intended to specify the presence of the stated features, elements, components, groups, integers, and/or steps as well as those that do not materially affect the basic and novel characteristic(s) of features, elements, components, groups, integers, and/or steps.

Terms of degree such as “substantially”, “about” and “approximately” as used herein mean a reasonable amount of deviation of the modified term such that the end result is not significantly changed. These terms of degree should be construed as including a deviation of at least ±5% of the modified term if this deviation would not negate the meaning of the word it modifies. In addition, all ranges given herein include the end of the ranges and also any intermediate range points, whether explicitly stated or not.

As used in this disclosure, the singular forms “a”, “an” and “the” include plural references unless the content clearly dictates otherwise.

In embodiments comprising an “additional” or “second” component, the second component as used herein is chemically different from the other components or first component. A “third” component is different from the other, first, and second components, and further enumerated or “additional” components are similarly different.

The term “and/or” as used herein means that the listed items are present, or used, individually or in combination. In effect, this term means that “at least one of” or “one or more” of the listed items is used or present.

The abbreviation, “e.g.” is derived from the Latin exempli gratia and is used herein to indicate a non-limiting example. Thus, the abbreviation “e.g.” is synonymous with the term “for example.” The word “or” is intended to include “and” unless the context clearly indicates otherwise.

It will be understood that any component defined herein as being included may be explicitly excluded by way of proviso or negative limitation, such as any specific compounds or method steps, whether implicitly or explicitly defined herein.

### II. Biosensors

Described herein are biosensors. The biosensors are useful for detecting the presence of a target molecule in a sample. Typically, the target molecule is a protein specific to a target microorganism, such as a bacterial target, but it is contemplated that any target molecule and thus microorganism could be used with the biosensors described herein.

The biosensors comprise a polymeric hydrogel, a colorimetric indicator entrapped in the polymeric hydrogel; and an oligonucleotide that crosslinks the polymeric hydrogel. When the target molecule is present, the oligonucleotide is cleaved or otherwise separated, such that the crosslinks are broken, thereby releasing the colorimetric indicator from the hydrogel.

Typically, the biosensors use a DNAzyme, which is an oligonucleotide with enzymatic activity. The DNAzyme is activated in the presence of the target molecule and then acts to reverse the crosslinks formed by the oligonucleotide, thereby breaking down the hydrogel and releasing the colorimetric indicator. The target molecule may act directly on the DNAzyme to activate it, or it may interact, for example, with an aptamer that normally blocks the enzymatic activity of the DNAzyme. Thus, when the target molecule is present, it removes the inhibitory effect of the aptamer on the DNAzyme, allowing its enzymatic activity to reverse the crosslinks in the hydrogel.

Thus, it will be understood that this platform is broadly applicable to any target molecule that is capable of interacting directly with a DNAzyme or indirectly, for example, via an aptamer.

Typically, the target molecule is a protein and, typically, the protein is specific to a microorganism. The biosensors described herein are particularly useful for detecting microorganisms, such as bacteria, that are responsible for food- or water-borne illnesses. Examples include E. coli, Salmonella, Listeria monocytogenes, Legionella pneumophila, Campylobacter, Shigella, Vibrio, and Staphylococcus aureus. Typically, the target is E. coli.

Other target microorganisms include fungal targets, such as Aspergillus, viral targets, such as Norovirus and Hepatitis A, protozoan targets, such as Cryptosporidium, Cyclospora, Entamoeba, and Giardia.

When the molecule is from a bacterial target, it has been described herein that a lytic bacteriophage that is capable of infecting and lysing the bacterial target may be included in the biosensor. This lyses the bacterial target, thereby increasing the concentration of the target molecule in the sample, particularly if the target molecule is an intracellular molecule. Any bacteriophage may be used, provided it is a lytic bacteriophage. For example, in the case of E. coli, Bacteriophage T7 is a typical choice. It will be understood that species of bacteriophage are numerous and the skilled person could readily select a lytic bacteriophage that is capable of infecting the bacterial species of interest.

The colorimetric indicator chosen for use in the biosensors described herein is generally something that is large enough to be trapped inside the polymeric hydrogel. Gold nanoparticles is a typical choice. Other examples include silver nanoparticles or blue dextran. The colorimetric indicator, in some cases, may be bulked to make it larger and harder to diffuse from the hydrogel until the crosslinks are reversed. For example, the colorimetric indicator may be coated with a substance that does not interfere with its color indicating abilities. For example, BSA may be coated on the surface of nanoparticles, such as gold nanoparticles, to make them bulkier.

In some aspects, the polymeric hydrogel comprises acrylamide and the oligonucleotide is acrydite-modified so that it will interact with the acrylamide, so that crosslinks may be formed. In some examples, the oligonucleotide is formed from two hybridizing strands, a substrate strand and a DNAzyme strand. Each strand is separately colpolymerized with a portion of acrylamide and when mixed together, the DNAzyme strand and the substrate strand hybridize together, thereby crosslinking the hydrogel. In this particular example, the DNAzyme strand cleaves the substrate strand in the presence of the target molecule.

As explained herein, any oligonucleotide specific to any target may be used. In the case of E. coli, typically the DNAzyme strand comprises the sequence GA TGT GCG TCT TGA TCG AGA CCT GCG ACA GGA AG (SEQ ID NO: 1), or a sequence at least 75%, 80%, 85%, 90%, 95%, or 97% identical thereto and the substrate strand comprises the sequence TT TTT ACT CTT CCT AGC TrAT GGT TCG ATC AAG A (SEQ ID NO: 2), or a sequence at least 75%, 80%, 85%, 90%, 95%, or 97% identical thereto.

The biosensors described herein are sensitive, being capable of detecting the presence of bacteria in an amount of at least about 101 CFU mL−1. The biosensors are also specific, with little risk of a false positive result. Moreover, the biosensors are quick, producing a visible result in less than about 18 hours.

Advantageously, while equipment may be used to assess or analyze the result, the result is colorimetric and a unskilled user can view the result with the naked eye. No equipment is needed for viewing or analysis of the test result.

The biosensors described herein are stable and can be stored for periods of time. For example, the biosensors are typically stable for at least two weeks at temperatures between about −20° C. and about 20° C.

The sample being tested is typically a liquid sample or a liquidized sample. For example, the sample may be a water sample, for example, from a drinking water source or from water sprayed on fruits or vegetables, a liquid food sample, or a bodily fluid sample. The sample may be from a food source, and/or a surface. In some cases, the sample is liquidized in order to make it suitable for testing, as described herein.

Also described herein are methods of detecting a target molecule in a sample, the method comprising applying the sample to the biosensor described herein. An AI network may be used to assess the colorimetric result in order to avoid any subjectivity.

Also described herein is a method of amplifying DNAzyme signalling in a biosensor that detects a bacteria-specific protein from whole bacteria, the method comprising applying to the sensor a lytic bacteriophage that infects and lyses the bacteria.

### EXAMPLES

The following non-limiting examples are illustrative of the present disclosure:

### Example 1: Bacteriophage-Activated DNAzyme Hydrogels Combined with Machine Learning Enable Point-of-Use Colorimetric Detection of Escherichia coli

### Abstract

Developing cost-effective, equipment-free platforms for point-of-use environmental and clinical pathogen testing is a priority, to reduce reliance on laborious, time-consuming culturing approaches. Unfortunately, a system offering ultrasensitive detection capabilities in a form that requires no auxiliary equipment or training has remained elusive. Here, we present a colorimetric DNAzyme-crosslinked hydrogel sensor. In the presence of a target pathogen, DNAzyme cleavage results in hydrogel dissolution, yielding the release of entrapped gold nanoparticles in a manner visible to the naked eye. Recognizing that Escherichia coli holds high relevance within both environmental and clinical environments, an E. coli-responsive DNAzyme was incorporated into this platform. Through the optimization of the hydrogel polymerization process and the discovery of bacteriophage-induced DNAzyme signal amplification, 101 CFU mL−1 E. coli was detected within real-world lake water samples. Subsequent pairing with an artificial intelligence model removed ambiguity in sensor readout, offering 96% true positive and 100% true negative accuracy. Finally, high sensor specificity and stability results supported clinical use, where 100% of urine samples collected from patients with E. coli urinary tract infections were accurately identified. No false positives were observed when testing healthy samples. Ultimately, this platform stands to significantly improve population health by substantially increasing pathogen testing accessibility.

### Introduction

With the emergence of antibiotic resistance, limiting pathogenic exposure has become a growing priority. Currently, one of the most defined mechanisms for such exposure involves the consumption of unsafe drinking water, with an estimated 1.8 billion people relying on water sources that are prone to fecal contamination.[1] A prevalent issue within low-income and rural communities, inadequate treatment of such water supplies prior to consumption yields significant risk of disease.[2] To this end, effective water monitoring is key, wherein Escherichia coli is often used as an indicator of water safety.[3,4] While not always pathogenic in nature, its prevalence in the digestive tract of humans and animals makes it an effective marker of fecal contamination. When infection does occur—irrespective of the source, rapid diagnosis is particularly important towards ensuring optimal patient outcomes. Here, E. coli is one of the leading causes of disease, accounting for an estimated 950K annual deaths globally.[5] Once again, severe ailments in this space disproportionally impact rural and low-income communities, due to a lack of effective testing resources. There is an established need for effective E. coli detection for both environmental monitoring and clinical diagnosis, particularly in low-income and remote communities.

While various approaches for bacterial detection are currently employed, conventional methods have requirements which limit their widespread applicability. For example, bacterial culturing, which remains the gold-standard approach for E. coli detection, is time-consuming, relies on trained personnel, and requires specialized laboratory equipment.[6,7] In an effort to alleviate these drawbacks, many on-site detection platforms have been proposed in literature.[8] This includes electrochemical,[9] enzymatic,[10,11] aptamer,[12,13] and antibody-based methods.[14,15] Despite promising offerings, no platform has offered high sensitivity and specificity towards E. coli in a form factor that is easy-to-distribute, simple-to-use, equipment-free, works with both urine and real-world water samples, and is faster than conventional approaches.

Given these requirements, bacteria-responsive RNA-cleaving DNAzymes are particularly promising for such on-site applications.[6,16] These molecules generally consist of an enzymatic strand hybridized to a substrate strand. Once a target binds to the enzymatic strand, its catalytic activity is activated, yielding cleavage at a ribonucleotide site located within the substrate strand. Owing to their ability to function as stable, standalone detection molecules, these molecules yield sensitive and specific biosensors that require limited handling and processing.[17] While this has inspired the development of several DNAzyme-based fluorescence biosensors for bacterial detection from our group, including for E. coli,[18-21] an equipment-free system must offer colorimetric signal transduction to enable sensor assessment with the naked eye.[17] To this end, previous works have detailed DNAzyme crosslinked hydrogels for the colorimetric detection of heavy metals using metal ion-specific DNAzymes.[22-24] Briefly, these hydrogels involve the entrapment of colored agents within a polymer chain network that is held together by target-responsive DNAzymes.[25] Gel dissolution in the presence of the target yields visible color release.

Importantly, while metal ion targets can readily diffuse through such hydrogel matrices to activate gel dissolution, bacterial targets are much larger in size. Fortunately, bacteria-responsive DNAzymes are not activated by whole cells, but rather by highly specific entities (i.e., proteins) secreted by the target cell. Yet, recognizing the extensive DNAzyme crosslinking that holds such a gel together, effective bacterial detection would require a significant concentration of the cleavage-inducing entity in the test solution. Introducing an equipment-free approach for bacterial lysis thus represents a promising approach toward signal amplification. That being said, both environmental and clinical samples are rich in microbes, meaning that targeted lysis is important to limit the unwanted presence of non-specific entities in the microscale hydrogel matrix.

Herein, we present a DNAzyme-based colorimetric hydrogel biosensor for the equipment-free detection of E. coli in complex samples. The biosensor includes a polyacrylamide hydrogel crosslinked with a novel E. coli-responsive DNAzyme construct that is activated by an E. coli-specific protein (ECP1). The hydrogel matrix is embedded with gold nanoparticles (AuNPs), that offer colorimetric transduction in response to ECP1-induced DNAzyme cleavage. The biosensor also incorporates Bacteriophage T7—an E. coli-infecting phage with high lytic activity,[26-28] to specifically maximize ECP1 availability, yielding signal amplification. Notably, this is the first report of bacteriophage-induced amplification of DNAzyme signalling. The resultant sensor offered highly specific and sensitive detection of E. coli, with concentrations as low as 101 CFU mL−1 inducing color shifts visible to the naked eye within 18 hours. The sensor also offered excellent stability in response to various environmental stressors, effectively maintaining its detection capabilities. When tested with real-world environmental water samples and clinical urine samples, application-relevant detection performance was observed. Finally, an artificial intelligence (AI) network was developed to automatically classify gels based on the degree of color shift, to improve system accessibility and remove user ambiguity. The network effectively classified tested biosensors as either contaminated or uncontaminated with a 96% true positive accuracy.

### Results and Discussion

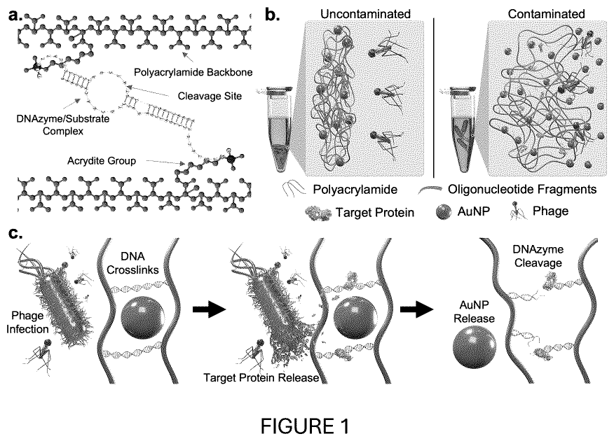

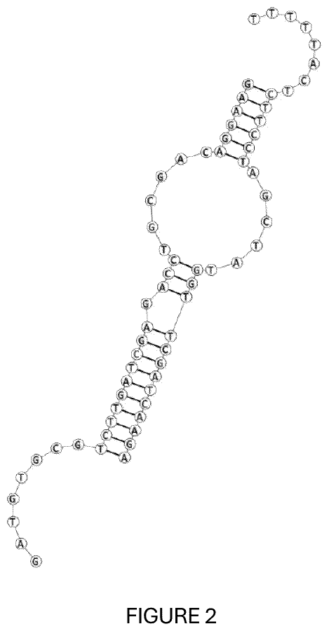

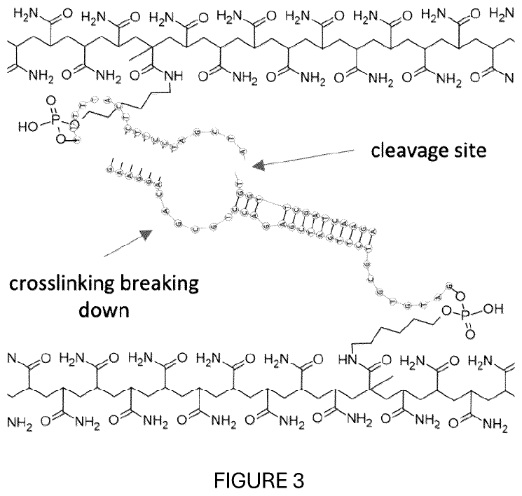

Fabrication and Design of the E. coli-responsive Hydrogel Biosensor: The developed hydrogel biosensor is composed entirely of polyacrylamide chains and oligonucleotides, wherein the polymer chains are held together by acrydite-modified, trans conformation E. coli-responsive DNAzymes (FIGS. 1a, 2, 3). This acrydite modification enables the copolymerization of the enzymatic and substrate oligonucleotides with acrylamide monomers during chain polymerization. The two sequences are individually copolymerized, and then mixed together to induce hybridization-based DNAzyme crosslinking (Table 1). AuNPs introduced during this step are entrapped within the gel matrix. As this study sought to develop a biosensor that could be used on-site by untrained users with no access to auxiliary equipment, the hydrogel biosensor was embedded within a tube (FIG. 1b). To this, the user can simply add their test sample, alongside pre-packaged reagents. When a test sample is added to the tube, it slowly permeates through the hydrogel sensing matrix, with significant excess of the added solution overlaying the gel. If E. coli is present in the tube, free-flowing ECP1 interacts with the DNAzyme, inducing a cleavage reaction that results in gel dissolution-driven AuNP release that is visible to the naked eye (FIG. 1c, 4).

TABLE 1
DNAzyme and substrate sequences with acrydite modifications.

| Component | Sequence |
|---|---|
| DNAzyme | /5 Acryd/GA TGT GCG TCT TGA TCG AGA CCT GCG ACA GGA AG (SEQ |
| | ID NO: 3) |
| Substrate | /5 Acryd/TT TTT ACT CTT CCT AGC TrAT GGT TCG ATC AAG A (SEQ ID |
| | NO: 4) |

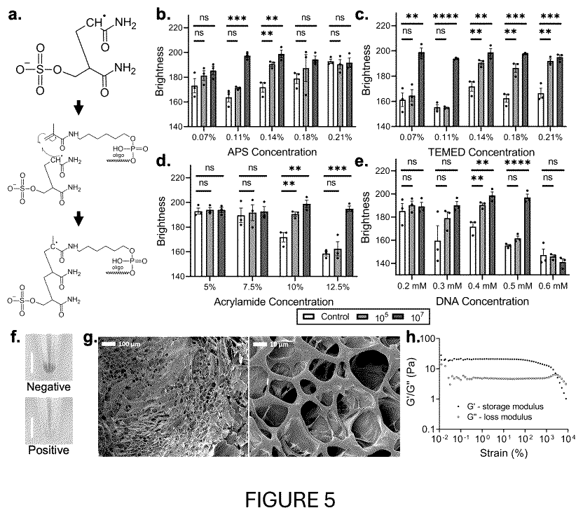

Optimization and characterization of polymer and hydrogel detection mechanism: The polymer components of the developed hydrogel detection platform were synthesized using free radical polymerization (FRP) (FIG. 5a). As mentioned above, two identical polymerizations were completed, with one containing the catalytic oligonucleotide strand and the other containing the substrate strand. The conditions under which an FRP reaction is performed dictate the characteristics of the resultant polymer products. When subsequently used to produce hydrogels, these characteristics influence the mechanical and chemical properties of the gel. In particular, the concentrations of the initiator, catalyst, and monomer dictate chain length and determine the viscosity of the resultant hydrogel.[29] With regards to sensing, this impacts the accessibility of the DNAzyme crosslinking sites, as well as the hydrogel's ability to effectively entrap colored agents. On the other hand, acrydite-modified DNA concentration determines the cross-linking density—a sensing parameter that needs to balance high hydrogel stability in the absence of the target, with efficient target-induced breakage.

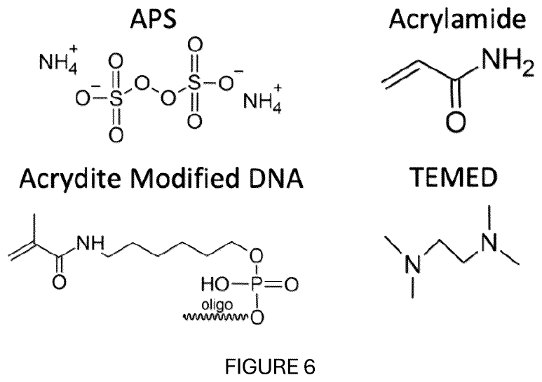

To maximize the performance of the E. coli-responsive hydrogel biosensor, the concentrations of each of these components (FIG. 6) were optimized. Specifically, hydrogels produced with varying reagent compositions were tested under both E. coli-positive (107 CFU mL−1, 105 CFU mL−1) and E. coli-negative conditions. Ammonium persulfate (APS) and N,N,N′,N′-Tetramethyl ethylenediamine (TEMED) were used as the initiator and catalyst, respectively, with both being evaluated at concentrations between 0.07% to 0.21% (FIG. 5b-c). When an APS concentration of 0.07% was employed, the fabricated hydrogels were not stable, as insufficient initiation yielded limited chain formation. While stable hydrogels were produced at an APS concentration of 0.11%, they did not respond to the presence of E. coli at a concentration of 103 CFU mL−1. Contrarily, an APS concentration of 0.14% yielded effective 103 CFU mL−1 E. coli detection. It is hypothesized that the lower frequency of initiation events that occur at an APS concentration of 0.11% yield longer polymers chains that impede ECP1 diffusion into the hydrogel matrix. When APS concentration was increased to 0.18% and 0.21%, the resultant hydrogels were mechanically weak, as excess initiation yielded short polymer chains unable to form a stable bulk material. This results in a significant risk of false positives, as AuNPs are not effectively entrapped within the polymer matrix. An APS concentration of 0.14% was thus considered optimal. With regards to TEMED, concentrations of 0.14%, 0.18%, and 0.21% all yielded hydrogels that offered optimal performance across the three test conditions. That being said, hydrogels produced using 0.14% TEMED offered the greatest mechanical stability, by drawing a balance between monomer accessibility for initiation and chain elongation rate. This concentration was thus selected.

In relation to the acrylamide monomer, concentrations of 5%, 7.5%, 10%, and 12.5% (w/v) were evaluated (FIG. 5d). Hydrogels could not be produced using monomer concentrations exceeding 12.5%, as the FRP products were too viscous for processing. At monomer concentrations of 5% and 7.5%, a solid hydrogel did not form, as sufficient chain length was likely not achieved. An increase in concentration to 10% resolved this issue, offering effective detection performance across all three test conditions. Further increasing the monomer concentration to 12.5% still yielded stable hydrogels, but 103 CFU mL−1 E. coli was not detected—likely due to increased DNAzyme inaccessibility caused by longer polymer chains. A 10% acrylamide monomer concentration was thus used within subsequent synthesis.

Finally, the concentration of acrydite-modified DNA was evaluated at concentrations between 0.2 mM and 0.6 mM (FIG. 5e). At concentrations of 0.2 and 0.3 mM, fabricated hydrogels lacked stability, yielding unreliable detection. Specifically, the reduced crosslinking density within these hydrogels caused spontaneous breakage in the absence of E. coli, resulting in false positive signalling. Stability was afforded with a DNA concentration of 0.4 mM, wherein effective detection was also observed. However, using DNA concentrations of 0.5 mM and 0.6 mM yielded hydrogels that were not responsive to 103 CFU mL−1 E. coli, as their increased crosslinking density requires higher ECP1 target availability for breakage. The 0.4 mM concentration was thus selected.

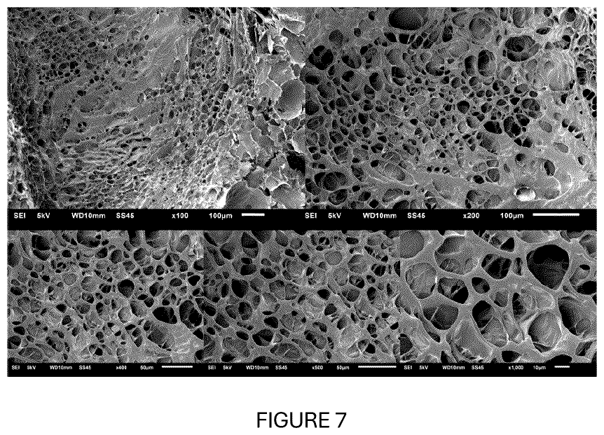

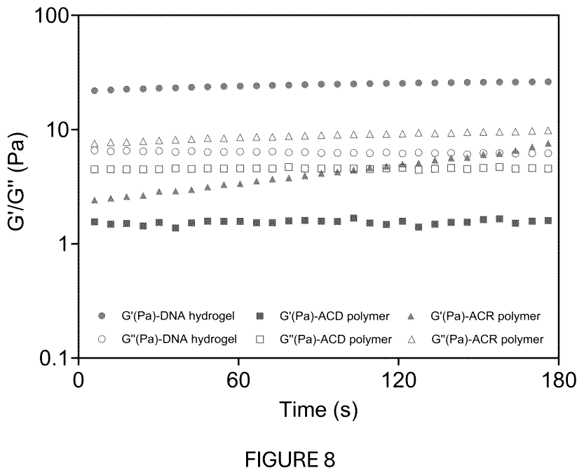

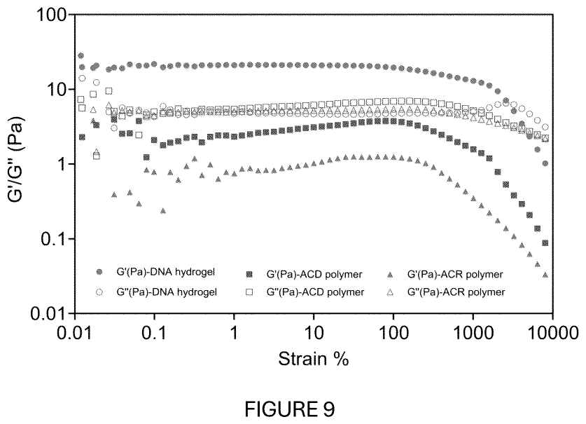

Collectively, these optimized parameters produced hydrogels with an appropriate trade-off between physical stability and detection performance (Table 2). The resulting signal was visible to the naked eye (FIG. 5f). These optimized hydrogels were visualized using scanning electron microscopy (SEM) and rheology. SEM was performed after freeze drying to preserve the hydrogel structure, wherein pores with diameters between 10 and 20 μm were observed (FIGS. 5g, 7). G′ (storage modulus) was greater than G″ (loss modulus) for the hydrogel rheology tests until shear thinning behaviour at strains exceeding 1000% (FIG. 5h). The same was not true for the individual polymer components before the enzymatic and substrate strands are mixed (FIGS. 8, 9), showing the gelation impact of hybridization.

TABLE 2
Polymerization reagent concentrations and quantities

| Reagent | Concentration | Volume (μL) |
|---|---|---|
| Acrylamide | 25% in water (w/v) | 50 |
| DNA (DNAzyme or Substrate) | 3 mM | 16 |
| Water | N/A | 50 |
| APS | 10% in water (w/v) | 1.68 |
| TEMED | 10% in water (v/v) | 1.68 |

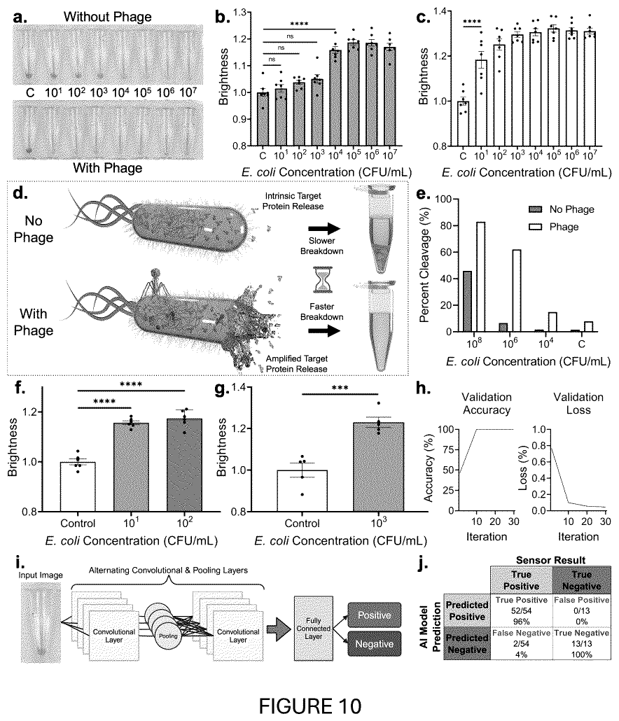

Bacteriophage and machine learning integration for environmental sample testing: With optimal polymerization parameters identified, the sensitivity of the sensor was tested through incubation with water samples contaminated with 101 to 107 CFU mL−1 E. coli (FIG. 10a). The limit of detection (LOD) was qualitatively and quantitatively determined to be 104 CFU mL−1 (FIG. 10b). While promising, this level of performance is not sufficient for many real-world applications. While culturing samples prior to testing would overcome this issue, such a step would both increase the assay time and limit on-site, untrained sensor use. It was hypothesized that a similar improvement in LOD could be enacted through the use of E. coli-specific lytic T7 bacteriophage.[30-32] Here, these agents would lyse and thus release all DNAzyme-activating proteins present within intracellular environments, significantly increasing cleavage activity without increasing assay time. This proposed cascade was explored using T7 bacteriophage, wherein the sensor's LOD was improved by several orders of magnitude to 101 CFU mL−1 (FIG. 10c), as confirmed by qualitative visual inspection and quantitative analysis (FIG. 10a, 11). Given that qualitative detection via visual inspection offered such high sensitivity, it can be noted that this sensor does not require any equipment for ultrasensitive sample analysis. This sensor thus has considerable potential towards on-site testing of environmental and clinical samples. The bacteriophage-mediated sensing mechanism is visualized in FIG. 10d.

To further validate the proposed bacteriophage amplification mechanism, direct cleavage tests were performed on non-crosslinked DNAzyme samples with and without T7 bacteriophage (FIG. 10e). Here, cleavage rate was quantified through gel electrophoresis, wherein bands formed by cleavage fragments provided a measure of percent cleavage in a given sample. Here, the samples with bacteriophage achieved a percent cleavage of 82.92% at 108 CFU mL−1 E. coli. By contrast, 108 CFU mL−1 E. coli samples without bacteriophage only had a percentage cleaved of 45.86%.

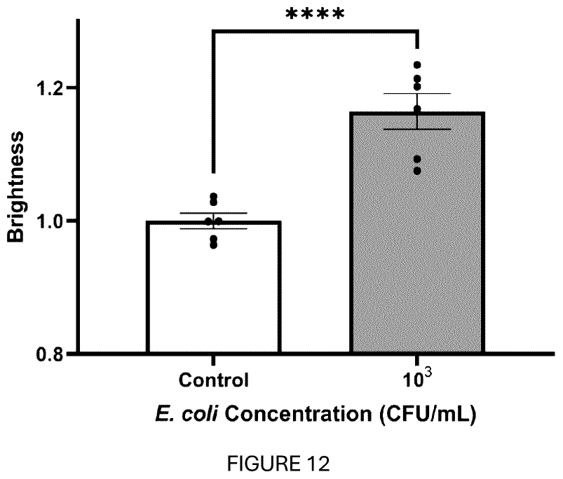

To assess the suitability of this platform for real-world environmental testing, lake water and cistern water—both prone to microbial contamination,[3,4,33] were spiked with low concentrations of E. coli and tested (FIG. 10f-g, Table 3). E. coli was successfully detected in both lake water and cistern water using this platform, at concentrations as low as 10¹ CFU mL⁻¹ and 10³ CFU mL⁻¹, respectively (FIG. 12).

TABLE 3
pH measurements of water sources used for experiments

| Water Type | Reading 1 | Reading 2 | Reading 3 | Average |
|---|---|---|---|---|
| Ultrapure Water | 7.60 | 7.27 | 7.29 | 7.39 |
| Lake Water | 8.01 | 8.05 | 8.07 | 8.04 |
| Cistern Water | 8.25 | 8.24 | 8.24 | 8.24 |

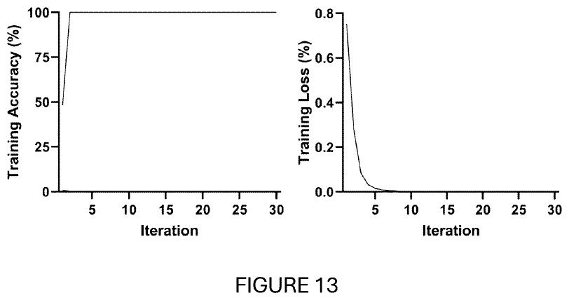

To further automate and increase the ease-of-use of this platform, a convolutional neural network (CNN) AI model was trained from the optical images (FIG. 10h, 10i). The strong image analysis capabilities of this network make it well suited for this application.[34] Images used to create the model were split randomly into training and validation data. After every 10 iterations of training the model with the training data, it was assessed with the rest of the images for validation. This resulted in a final validation accuracy of 100% for the model. Training accuracy and loss plots are shown in FIG. 13. The model was then assessed using a separate testing dataset that included images of sensors exposed to different concentrations of E. coli, ranging from 101 to 107 CFU mL−1. This final assessment resulted in a true positive rate of 96% and true negative rate of 100%, as shown in a confusion matrix (FIG. 10j). By enabling automated sensor readout using a simple optical image, this AI model effectively eliminates ambiguity surrounding colorimetric readout, further increasing the accessibility and ease-of-use of the developed sensor.

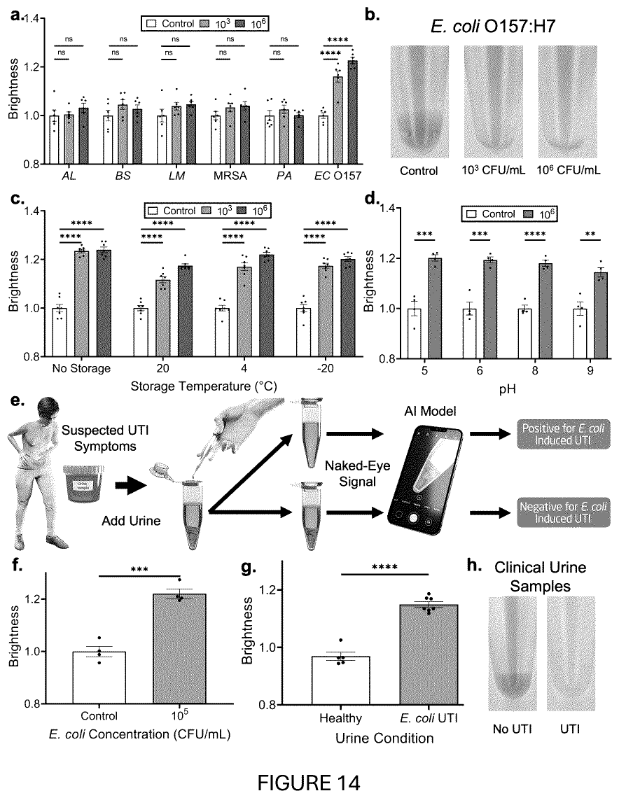

Sensor specificity, stability, and performance against clinical urine samples: Next, the specificity of the developed sensor was tested. Here, E. coli O157:H7 was used as a positive test sample, while Acinetobacter lwoffii, Bacillus subtilis, Listeria monocytogenes, Pseudomonas aeruginosa, and methicillin-resistant Staphylococcus aureus were used as non-specific test samples (FIG. 14a-b). 103 and 106 CFU mL−1 bacterial suspensions were employed for each bacterial condition. While a statistically significant signal was observed for both concentrations of E. coli O157:H7, the same was not true for the other bacterial species. This affirms that the trans confirmation of the EC1 DNAzyme employed in this work maintains the high degree of specificity offered by its previously reported cis counterpart.[6,35] The retainment of E. coli O157:H7-responsive activity is particularly important, as detection of this pathogen ensures high value applicability within various environmental testing circumstances.[36]

The stability of the developed sensor within diverse long-term storage conditions was then assessed. Sensors were stored at temperatures of −20° C., 4° C., and 20° C. for two weeks and then tested using uncontaminated, 103 CFU mL−1 E. coli, and 106 CFU mL−1 E. coli test samples (FIG. 14c). Detection performance was compared to that of sensors tested immediately after fabrication. While sensors stored at −20° C. most effectively maintained colorimetric signal intensity in terms of visual appearance, sensors from all storage conditions offered accurate E. coli detection. These results further supported the commercial feasibility of the developed sensor.

Next, to evaluate the viability of the developed sensor for the analysis of acidic and basic test samples, the impact of test sample pH on sensor performance was assessed. Water samples with pH levels ranging from 5 to 9 were used to test sensors in both an uncontaminated state and a contaminated state, wherein E. coli loads of 103 and 106 CFU mL−1 were employed (FIG. 14d). The sensor retained its ability to accurately report both the absence and presence of E. coli under all tested concentrations. Such pronounced stability across a wide pH range suggests that the developed platform may be suitable for the evaluation of a wide range of test sample types. In particular, these results offered support towards clinical sample analysis.

To comprehensively evaluate such a premise, proof-of-concept testing was performed using urine samples, where effective E. coli monitoring offers significant value towards UTI diagnosis (FIG. 14e).[37,38] To account for any non-specific urine-induced signals, healthy urine was used as a negative control for E. coli-spiked urine samples. Successful detection of the E. coli infection was observed, wherein all test samples offered a significantly higher signal than all control samples (FIG. 14f). Finally, healthy and infected clinical urine samples collected from local healthcare facilities were tested using the developed sensor to account for any differences in real-world infection. Here, seven E. coli-infected samples and five E. coli-unrelated samples were tested (FIG. 14g-h, 15 and Table 4). Accurate assessment was enacted with all patient samples, with detection interpretable to the naked eye (FIG. 14h).

TABLE 4
pH and infection status of clinical urine samples.

| Sample | | |
|---|---|---|
| Number | UTI Status | Sample pH |
| 1 | E. coli Infection (>10⁵ CFU/mL) | 5.26 |
| 2 | E. coli Infection (>10⁵ CFU/mL) | 5.10 |
| 3 | E. coli Infection (>10⁵ CFU/mL) | 6.49 |
| 4 | E. coli Infection (>10⁵ CFU/mL) | 4.91 |
| 5 | E. coli Infection (>10⁵ CFU/mL) | 6.07 |
| 6 | E. coli Infection (>10⁵ CFU/mL) | 6.21 |
| 7 | E. coli Infection (>10⁵ CFU/mL) | 5.79 |
| 8 | No Infection | 5.53 |
| 9 | No Infection | 6.73 |
| 10 | No Infection | 5.00 |
| 11 | No Infection | 6.11 |
| 12 | No Infection | 6.89 |

Conclusion: An ultrasensitive hydrogel detection platform that can be applied to water and urine testing has been created. Due to its colorimetric signal that can be interpreted with the naked eye and its pH and temperature stability, this sensor is well suited to real-world on-site testing of a variety of potentially contaminated water sources as well as for at-home UTI testing. The CNN AI model that has been developed enables the automatic classification of samples into the categories of contaminated or uncontaminated, based only on optical images. By using T7 bacteriophage for lysis, the sensitivity of the developed sensor has been improved by several orders of magnitude. The phage-induced improvement to sensor performance reported in this work is notable even separate to this specific platform. This observation potentially has wide-ranging applicability to other DNAzyme-based bacterial sensing platforms, where phage may also be useful as an equipment-free method of inducing bacterial lysis for signal amplification. Comprehensive assessments of such phage-DNAzyme interactions represent a key area of study for future works. Subsequent works should also explore crosslinking the developed hydrogel sensors with DNAzymes specific to various bacterial targets, to increase the use-case of the developed platform. Ultimately, this E. coli sensing platform has immediate relevance both for real-world water and urine testing applications, while also providing a foundation for further research into colorimetric bacteria-detecting hydrogels and phage-amplified sensing.

### Methods

Materials: 0.2 mL clear flat cap PCR tubes were acquired from Diamed Lab Supplies Inc (Ontario, Canada). Ammonium persulfate (APS), N,N,N′,N′-Tetramethyl ethylenediamine (TEMED), 40% acrylamide monomer solution, MgCl2 buffer, gold chloride solution, trisodium citrate dihydrate, and bovine serum albumin (BSA) were purchased from Millipore Sigma (Ontario, Canada). Bacto™ yeast extract was purchased from ThermoFisher Scientific (Ontario, Canada). Acrydite modified oligonucleotides were ordered from Integrated DNA Technologies (IDT) (Iowa, USA).

Bacterial Preparation: E. coli K12, E. coli O157:H7, A. lwoffii, B. subtilis, L. monocytogenes, P. aeruginosa, and methicillin-resistant S. aureus were each cultured from glycerol stocks in a shaking incubator for 18 hours at 37° C. and 180 RPM in suitable media. The culture solutions were then centrifuged at 7000 RCF for 15 minutes at 4° C., and the resulting bacterial pellet was resuspended in DI water.

Nanoparticle Synthesis and BSA Coating: A modified version of the procedure described by Grabar et al. was used.[39] To synthesize the gold nanoparticles, 200 uL of gold chloride solution was added to 300 mL of DI water in an Erlenmeyer flask with a magnetic stir bar. This solution was stirred at 1200 RPM and heated to 100° C., monitored with a temperature probe. A separate solution of 0.5 g trisodium citrate dihydrate in 30 mL of DI water was then added to the flask, with mixing continuing for 10 minutes. The Erlenmeyer flask was removed from heat and placed in an ice bath, with the solution still being stirred at 1200 RPM for an additional 15 minutes. To coat the nanoparticles in BSA, 15 mL of the nanoparticle solution was combined with 5 mL of 2% BSA and stirred for 18 hours at 300 RPM. The mixture was then centrifuged at 14 000 RPM for 15 minutes at 4° C., at which point the supernatant was removed and the particles were resuspended in DI water.

Phage propagation & filtering: T7 phage was propagated using a host, Escherichia coli strain K12 BL21 (Sigma-Aldrich, CMC001). Briefly, Aa pre-culture of E. coli in LB-Miller broth was cultured overnight in a shaking incubator (180 rpm at 37° C.). The next day, using fresh LB-Miller broth, a 1:200 subculture was prepared and allowed to grow until an OD600 nm of 0.6 was reached, upon which 10 μL of T7 phage (~1010 PFU/mL) was added. 50 μL of 1M CaCl2) solution was added to the subculture to assist phage infectivity. The T7-subculture solution was incubated in a shaking incubator (180 rpm at 37° C.) for 6 hours. The culture was then centrifuged at 7000×g for 15 minutes. The bacteria pellets were discarded, and the phage-containing supernatant was retained and filtered through a 0.2 μm filter (Fisher Scientific, 13100106) to remove residual bacteria. Filtered supernatant was stored at 4° C.

Double Overlay Phage Titer Assay: T7 phage was quantified using agar overlay technique. Briefly, 200 μL of E. coli K-12 BL21 and 100 μL of diluted T7 phage solution was added to liquefied LB-Miller Soft Agar (2.5% LB; 0.6% Agar), vortexed, and poured on top of LB-Miller Agar (2.5% LB; 1.5% Agar) plates (Fisher Scientific, Sterile 100 mm×15 mm Polystyrene Petri Dishes, FB0875712). Plates were placed in stationary incubator set at 37° C. for 6 hours and then incubated at room temperature for 12 hours. The number of resulting plaques were counted, and a total concentration was determined using the following equation:

$$Concentration = \frac{\# of plaques}{dilution factor \cdot phage volume added}$$

Denaturing Polyacrylamide Gel: An 8 M urea 10% polyacrylamide gel was prepared in a Tris/Borate/EDTA (TBE) buffer system. 18 μl of quenched cleavage reactions were heated at 90° C. for 1 minute, cooled at ambient temperature for 5 minutes and applied to the gel. Gels were run for a sufficient time to resolve cleavage products of EC1 from full-length sequence. Using the Cy2 filter set on a Cytiva Typhoon biomolecular imager (Center for Microbial Chemical Biology, McMaster University), electrophoresed gels were visualized by fluorescence scan. Image files were analysed using ImageJ.[40]

Polymerization: A modified version of the polymerization procedure described by Lin et al was used.[24] Two separate solutions each containing acrylamide monomer and one of the acrydite modified oligonucleotide sequences were heated to 45° C. Free radical polymerization was induced with the addition of APS and TEMED, at which point the tubes containing the solutions were put in a vacuum desiccator for 30 minutes. Final reagent concentrations are available in table 2.

Gold Nanoparticle Addition: 8 mL of the BSA coated gold nanoparticles in DI water were centrifuged at 14 000 RPM for 15 minutes and the supernatant was removed. 40 uL of this remaining solution was added to each tube of polyacrylamide-DNA solution to give them a red color, as well as 160 uL of DI water which was left for 24 hours and then thoroughly mixed.

Gel Fabrication: 0.2 mL PCR tubes were placed in a heat block at 95° C. 3 uL of each of the two polyacrylamide-DNA solutions were added to all of the PCR tubes, for a total volume of 6 uL. The complementary parts of the oligonucleotides result in DNA crosslinking of the polyacrylamide chains, causing gelation. The resulting hydrogels were then gradually cooled to room temperature in a PCR machine over 30 minutes, and the condensation settled at 8500 RPM using a VWR mini-centrifuge. Smaller 2 uL hydrogels were then taken by pipette from these larger gels and used for all experiments described, excluding the optimization experiments for which the full 6 uL gels were used.

Gel Optimization: E. coli K12 contaminated water, T7 bacteriophage, and MgCl2 buffer were added to each hydrogel and left to incubate for 2 days at 40° C. and 180 RPM. Gels were imaged and quantified as described in the following section.

Gel Sensitivity Testing: 15 uL of MgCl2 buffer was added to each hydrogel 24 hours before sensitivity testing. Resuspended E. coli K12 was serially diluted in DI water to concentrations from 101 to 107 CFU mL−1. 70 uL of these dilutions were added to each gel, along with 15 uL of an autolyzed yeast extract solution at a concentration of 50 mg mL-1. The tubes containing the hydrogel, buffer, yeast extract, and bacteria were then incubated at 40° C. and 180 RPM. After 6 hours 70 uL of T7 bacteriophage was added, and after 18 hours the samples were removed from the shaker and imaged on an Epson Perfection V850 Pro scanner. Results were quantified by measuring the mean brightness of a consistent area of each tube (50 pixels in height and 100 pixels in width) with ImageJ software.

Gel Specificity Testing: E. coli O157:H7, A. lwoffii, B. subtilis, L. monocytogenes, P. aeruginosa, and methicillin-resistant S. aureus were serially diluted in DI water to concentrations of 103 and 106 CFU mL−1 and tested under the same conditions as the sensitivity experiment.

Gel Stability Assessment: Prepared gels were stored in a sealed container at −20, 4, and 20° C. for 2 weeks, corresponding to storage in the freezer, fridge, and at room temperature. After storage the gels were tested according to the normal procedure with E. coli K12 concentrations of 103 and 106 CFU mL−1 and then imaged.

Environmental Samples: Lake water was collected in knee-depth water from Lake Erie near Dunnville, Ontario. Cistern water was collected from a cistern on a rural property also in Dunnville, Ontario.

Urine Testing: Urine samples were acquired from Hamilton General Hospital in Hamilton Ontario, Canada. Samples were collected according to the McMaster Research Ethics Board, with consent. The REB number is 2062. Of these 12 clinical samples, 7 were positive for E. coli (>105 CFU mL−1) and 5 were negative (Table 4). Urine samples were mixed with DI water at a ratio of 1:99, then tested with the platform following the procedure described in the ‘Gel Sensitivity Testing’ methods section. For the spiked urine experiment, the E. coli negative clinical sample listed as sample 13 in Table 4 was spiked with E. coli to a concentration of 105 CFU mL−1.

Freeze Drying and SEM: DNAzyme hydrogels were incubated overnight at −80° C., then freeze dried for 24 hours at −51° C. under 0.11 mBar vacuum. Following freeze drying, the gels were attached to the SEM stub with double sided carbon tape. After being coated with 15 nm gold (Quorum 300T D Plus sputter coater) the samples were imaged on the JEOL6610 LV SEM. 200× and 1000× magnification images were taken with secondary electron imaging (SEI) technique. Voltage was 5 kV. This took place at McMaster University (Ontario, Canada) at the Canadian Centre for Electron Microscopy (CCEM).

Rheology: Rheology Peltier setup parameters were used as follows: cone-plate geometry with 20 mm diameter, 1° cone angle, truncation gap 45 μm, sample amount 70 μL, test temperature 25° C. Mineral oil was applied around the circumference of the plates to prevent evaporation. A time sweep was carried out for 3 minutes at a fixed strain of 1% and frequency of 1 Hz. The strain sweep was completed from 0.01 to 10 000% strain at a fixed frequency of 1 Hz, 10 points per decade.

Artificial Intelligence Image Analysis: MATLAB's MathWorks Deep Learning Toolbox was used to train and test the CNN model. CNNs contain alternating convolutional and pooling layers, as well as a fully connected layer that decides the output according to the calculated probability of an image belonging to each output class (in this case, being positive or negative for E. coli). 88 images were used with a 70/30 training to validation split. Images were cropped to consistent size of 1000 by 400 pixels with one tube in each image. The model was trained and validated over 30 iterations, ending with a final validation accuracy of 100%. Training accuracy/loss plots are shown in FIG. 13.

### CITATIONS

[1] R. Bain, R. Cronk, R. Hossain, S. Bonjour, K. Onada, J. Wright, H. Yang, T. Slaymaker, P. Hunter, A. Pruss-Ustun, J. Bartram, Trop. Med. Int. Health 2014, 19, 917.
[2] P. Gwimbi, M. George, M. Ramphalile, Environ. Health Prev. Med. 2019, 24.
[3] Y. A. Pachepsky, D. R. Shelton, Crit. Rev. Environ. Sci. Technol. 2011, 41, 1067.
[4] D. D. Cloutier, S. L. McLellan, Appl. Environ. Microbiol. 2017, 83.
[5] K. S. Ikuta, L. R. Swetschinski, G. R. Aguilar, F. Sharara, T. Mestrovic, A. P. Gray, N. D. Weaver, E. E. Wool, C. Han, A. G. Hayoon, The Lancet 2022, 400, 2221.
[6] M. M. Ali, S. D. Aguirre, H. Lazim, Y. Li, Angew. Chem. Int. Ed. 2011, 50, 3751.
[7] M. R. Nurliyana, M. Z. Sahdan, K. M. Wibowo, A. Muslihati, H. Saim, S. A. Ahmad, Y. Sari, Z. Mansor, J. Phys. Conf Ser. 2018, 995, 012065.
[8] S. Khan, J. K. Monteiro, A. Prasad, C. D. Filipe, Y. Li, T. F. Didar, Adv. Mater. 2024, 36, 2300875.
[9] N. Razmi, M. Hasanzadeh, M. Willander, O. Nur, Biosensors 2020, 10, 54.
[10] S. Tok, K. Haan, D. Tseng, C. F. Usanmaz, H. C. Koydemir, A. Ozcan, Lab. Chip 2019, 19, 2925.
[11] N. S. K. Gunda, S. Dasgupta, S. K. Mitra, PLoS One 2017, 12, 0183234.
[12] Y. Yao, G. Xie, X. Zhang, J. Yuan, Y. Hou, H. Chen, Anal. Methods 2021, 13, 2209.
[13] R. Gupta, A. Kumar, S. Kumar, A. K. Pinnaka, N. K. Singhal, Sens. Actuators B Chem. 2021, 329, 129100.
[14] I. P. Alves, N. M. Reis, Biosens. Bioelectron. 2019, 145, 111624.
[15] F. Petronella, D. Biase, F. Zaccagnini, V. Verrina, S.-I. Lim, K.-U. Jeong, S. Miglietta, V. Petrozza, V. Scognamiglio, N. P. Godman, D. R. Evans, M. McConney, L. Sio, Environ. Sci. Nano 2022, 9, 3343.
[16] L. Gong, Z. Zhao, Y.-F. Lv, S.-Y. Huan, T. Fu, X.-B. Zhang, G.-L. Shen, R.-Q. Yu, Chem. Commun. 2012, 51, 979.
[17] S. Khan, B. Burciu, C. D. M. Filipe, Y. Li, K. Dellinger, T. F. Didar, ACS Nano 2021, 15, 13943.
[18] H. Yousefi, M. M. Ali, H.-M. Su, C. D. M. Filipe, T. F. Didar, ACS Nano 2018, 12, 3287.
[19] H. Yousefi, S. E. Samani, S. Khan, A. Prasad, A. Shakeri, Y. Li, C. D. M. Filipe, T. F. Didar, ACS Nano 2021, 16, 29.
[20] A. Prasad, S. Khan, J. K. Monteiro, J. Li, F. Arshad, L. Ladouceur, L. Tian, A. Shakeri, C. D. M. Filipe, Y. Li, T. F. Didar, Adv. Mater. 2023, 35, 2302641.
[21] S. Khan, Z. Hosseinidoust, Y. Li, C. D. Filipe, T. F. Didar, Nat. Rev. Bioeng. 2024, 1.
[22] Y. Huang, Y. Ma, Y. Chen, X. Wu, L. Fang, Z. Zhu, C. J. Yang, Anal. Chem. 2014, 86, 11434.
[23] Y. Huang, X. Wu, T. Tian, Z. Zhu, H. Lin, C. Yang, Sci. China Chem. 2017, 60, 293.
[24] H. Lin, Y. Zou, Y. Huang, J. Chen, W. Y. Zhang, Z. Zhuang, G. Jenkins, C. J. Yang, Chem. Commun. 2011, 47, 9312.
[25] S. Lilienthal, Z. Shpilt, F. Wang, R. Orbach, I. Willner, Acs Appl. Mater. Interfaces 2015, 7, 8923.
[26] L. Tian, K. Jackson, L. He, S. Khan, M. Thirugnanasampanthar, M. Gomez, F. Bayat, T. F. Didar, Z. Hosseinidoust, Nat. Protoc. 2024, 1.
[27] F. Bayat, A. Hilal, M. Thirugnanasampanthar, D. Tremblay, C. D. Filipe, S. Moineau, T. F. Didar, Z. Hosseinidoust, Nat. Commun. 2024, 15, 5626.
[28] L. Tian, L. He, K. Jackson, A. Saif, S. Khan, Z. Wan, T. F. Didar, Z. Hosseinidoust, Nat. Commun. 2022, 13, 7158.
[29] R. S. Shatat, S. K. Niazi, A. Ariffin, IOSR J. Appl. Chem. 2017, 10, 67.
[30] S. D. Ganeshan, Z. Hosseinidoust, Antibiotics 2019, 8, 131.
[31] F. Bayat, T. F. Didar, Z. Hosseinidoust, Environ. Sci. Nano 2021, 8, 367.
[32] H. M. Nguyen, C. Kang, J. Virol. 2014, 88, 2107.
[33] J. M. Baird, R. Summers, R. Plummer, Can. Water Resour. J. 2013, 38, 121.
[34] S. M. Anwar, M. Majid, A. Qayyum, M. Awais, M. Alnowami, M. K. Khan, J. Med. Syst. 2018, 42, 226.
[35] Z. Zhou, Y. Zhang, M. Guo, K. Huang, W. Xu, Biosens. Bioelectrics 2020, 167, 112475.
[36] S. E. Hrudey, P. Payment, P. M. Huck, R. W. Gillham, E. J. Hrudey, Water Sci. Technol. 2003, 47, 7.
[37] B. Foxman, Infect. Dis. Clin. 2014, 28, 1.
[38] G. Kahlmeter, J. Antimicrob. Chemother. 2003, 51, 69.
[39] K. C. Grabar, R. G. Freeman, M. B. Hommer, M. J. Natan, Anal. Chem. 1995, 67, 735.
[40] C. A. Schneider, W. S. Rasband, K. W. Eliceiri, Nat. Methods 2012, 9, 671.

### Example 2: Point-of-Use Colorimetric Detection of Escherichia coli in Food Matrices with DNAzyme Crosslinked Hydrogels

### Introduction

Foodborne pathogens continue to be major causes of illness, in North America and around the world. The World Health Organization has estimated that foodborne hazards resulted in 600 million illnesses in 2010, and 420 000 deaths.1 This includes contamination with pathogenic strains of Escherichia coli, such as E. coli 0157. Pathogenic E. coli strains have been known to contaminate various categories of food including meat, dairy, and produce such as leafy greens, sometimes resulting in extensive recalls.2-5 While food recalls are often only instituted after reports of illness, this retroactive detection can result in a delayed response and thus more widespread infection. In light of this, regular monitoring at various stages of the food supply chain is key for the detection of pathogenic bacteria before contaminated products are consumed.

Conventional methods of pathogen detection in food include bacterial culturing, as well as strategies such as polymerase chain reaction (PCR). However, these approaches typically require equipment, user training, and a laboratory environment, limiting their suitability for on-site testing applications. This renders them impractical for some stages of the food supply chain, such as at the retail or consumer level.

To address the general need for on-site bacterial testing, numerous pathogen sensing platforms have been developed. This can include immunoassays, enzymatic assays, nucleic acid-based methods including loop-mediated isothermal amplification, and electrochemical detection methods such as impedance flow cytometry.6-8 However, not all of these on-site platforms are compatible with food testing. The complex nature of food matrices may interfere with some biosensors through non-specific binding, sensor fouling, or other mechanisms.8,9 In particular, the high prevalence of non-target bacteria in many food samples poses a challenge for sensors that lack bacterial species specificity.

Our group has previously reported a hydrogel biosensor for the on-site colourimetric detection of E. coli.10 The hydrogel is made up of polyacrylamide, and is crosslinked together with a type of catalytic nucleic acid called DNAzymes. RNA-cleaving DNAzymes have a substrate strand and an enzymatic strand, which cleaves the substrate in response to a specific target.11 For this sensing platform, a DNAzyme responsive to a target protein from E. coli was chosen.12 Upon encountering the target protein, the DNAzyme crosslinking holding together the polyacrylamide hydrogel breaks down as the substrate strand is cleaved, and the gel degrades. This allows for the highly sensitive and specific detection of E. coli in an equipment-free colourimetric platform. In our previous work, however, the effectiveness of the sensing platform was only demonstrated for water and urine samples.

Herein, we demonstrate the compatibility of our hydrogel E. coli sensor with a wide range of food samples. The sensing platform performed colourimetric, naked-eye detection with samples from contaminated milk, produce, and ready-to-eat (RTE) food products in 18 hours. Detection has also been demonstrated with a known vector of E. coli, contaminated leafy greens. This widespread applicability to food products at risk of E. coli contamination increases the real-world relevance of our biosensing platform for infection prevention.

### Results and Discussion

### Design and Functionality of the Biosensing Platform

The basic design of our sensing platform has been described in our previous publication.10 It consists of polyacrylamide chains in which the acrylamide monomers have been co-polymerized with acrydite-modified oligonucleotides during free-radical polymerization. This results in the oligonucleotide strands, which are an E. coli responsive DNAzyme and substrate pair, being grafted to the polymer backbone. Complementary binding between the DNAzyme and substrate oligonucleotides crosslinks the polymer together to form a hydrogel, and bovine serum albumin (BSA) coated gold nanoparticles (AuNPs) are entrapped in the polymer matrix to achieve a visible red colour.

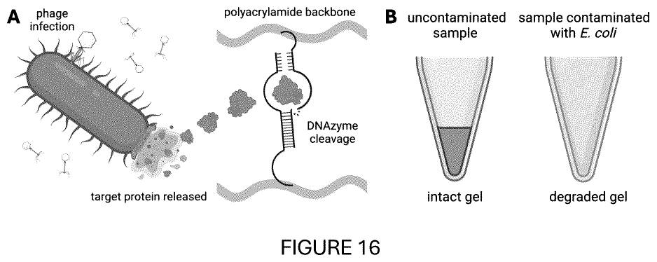

To function, the hydrogel sensor is combined with buffer, growth media, a potentially E. coli contaminated liquid sample, and T7 bacteriophage. This species of bacteriophage targets E. coli, inducing cell lysis in the infected bacteria and thus increasing the amount of target protein available to the sensor. If E. coli is not present in the sample, the gel will remain intact. However, if E. coli is present, the target protein will interact with the DNAzyme-substrate complex (FIG. 16A). This induces cleavage of the oligonucleotide crosslinking holding the gel together, and thus the degradation of the gel matrix. The difference between an intact and degraded gel can be seen on a macroscale with the naked eye (FIG. 16B). The visual result has also been quantified with ImageJ to produce the graphs seen in this publication.

Sensor Functionality with a Range of Food Samples

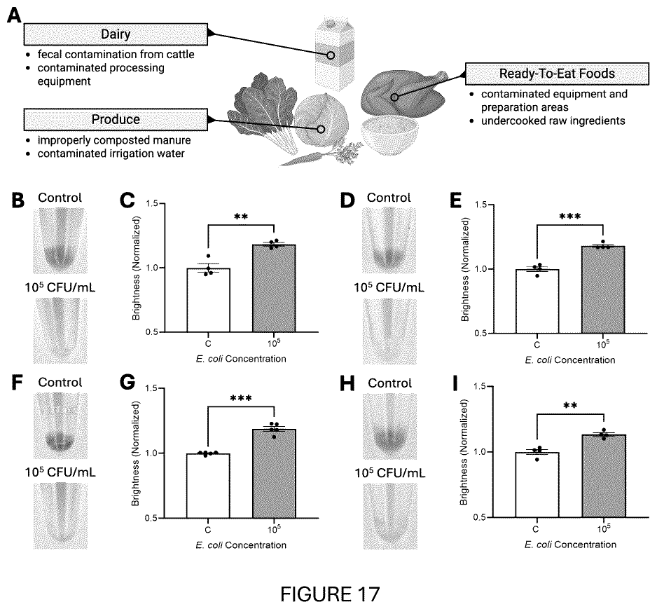

As previously mentioned, E. coli contamination can occur in a wide variety of food products including dairy products, produce, meat, and RTE foods (FIG. 17A). E. coli is ubiquitous in the digestive system of cattle, with some strains being enteropathogenic to humans.2 Thus, fecal contamination of dairy processing equipment can result in the presence of E. coli in unpasteurized or improperly pasteurized milk. Contamination of produce with pathogenic E. coli is also a concern, especially as vegetables are frequently eaten raw. This is often due to contaminated soil, irrigation water, or manure that has been inadequately composted.3,13 Leafy greens are a particular concern, and have been estimated by some to be responsible for around half of reported foodborne disease outbreaks in developed countries.14 While raw meat, in particular beef, is known to be a vector for pathogenic E. coli, the bacteria is typically killed during the cooking process.4,15 However, bacteria may remain if the cooking temperature is not adequate. Undercooked raw ingredients such as these are one pathway for E. coli contamination of RTE food products, in addition to improperly sterilized equipment and food preparation areas.

To address these applications, the sensing platform has been tested with several foods. First, milk was spiked to a concentration of 105 CFU mL−1 E. coli and tested with the sensing platform as described in the methods section. The first iteration of the test was unsuccessful, as the viscosity of the solution was too high for sensor functionality. To mediate this, the spiked milk was diluted as described in the methods before being added to the sensor. Here, E. coli was successfully detected after 18 hours at 37° C. The signal can be interpreted optically with the naked eye (FIG. 17B) and has also been quantified with ImageJ to show statistical significance (FIG. 17C).

Next, E. coli detection in RTE foods was assessed, including rotisserie chicken and hard boiled eggs. Chicken purge from a grocery store RTE rotisserie chicken was spiked with 105 CFU mL−1 E. coli, which was then detected with our sensing platform both optically (FIG. 17D) and graphically (FIG. 17E). Similar to the milk experiment, dilution of the spiked chicken purge was necessary due to viscosity challenges. The RTE eggs were individually packaged suspended in liquid, which was extracted and spiked with E. coli. Successful qualitative and quantitative detection at 105 CFU mL−1 was also achieved for these samples (FIG. 17F, 17G). Since RTE foods are intended for consumption without further washing or cooking steps, detection of bacterial contamination in these types of products can be a last line of defence before pathogen exposure.

To assess the functionality of the sensing platform with produce, the liquid from a bag of baby-cut carrots was extracted and spiked to a concentration of 105 CFU mL−1 E. coli. Like with the previously described food samples, this was successfully detected visually and when quantified (FIG. 17H, 17I). Carrots have been known to experience E. coli contamination, including during a notable 2024 recall across North America that sickened 48 with E. coli O121:H19.16 Thus, the sensor compatibility with carrots has a strong relevance for future real-world applications.

In summary, the demonstrated functionality of our sensing platform with a range of food products is critical for its practical use. The versatility of the platform makes it suitable for use at the general retail and consumer level, where using a different type of E. coli sensor for each category of food would likely not be feasible or practical.

Sensor Functionality with Leafy Greens

While several food products of interest have been discussed, E. coli contamination of leafy greens specifically is a notable public health issue.17 In light of this, the compatibility of the sensor with this type of food product was tested using multiple approaches for sample extraction.

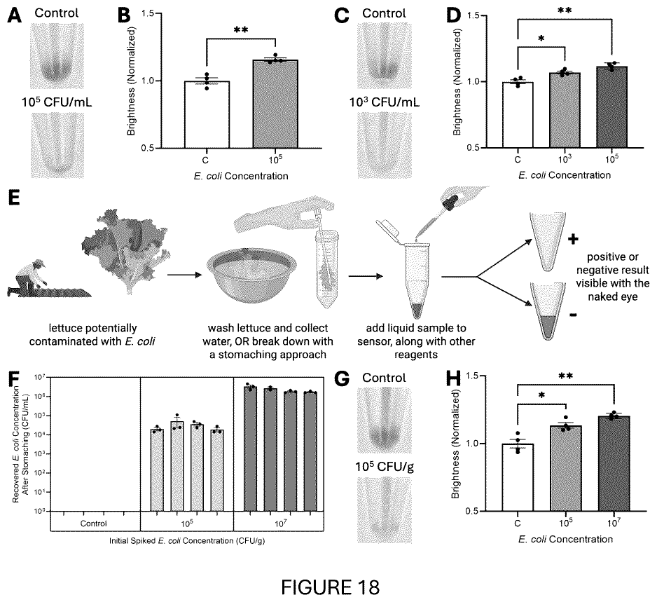

In many grocery stores, the produce section is frequently misted with water to maintain optimal humidity. The resulting droplets of water from the surface of a head of iceberg lettuce were collected, and spiked with 105 CFU mL−1 E. coli. The contaminated samples were successfully identified by the sensing platform, as shown visually (FIG. 18A) and on the graph (FIG. 18B). However, extracting a liquid sample from lettuce in such a way may not be practical for real-world applications. Therefore, we subsequently explored other methods of extracting liquid samples from lettuce for bacterial detection.

As it is customary for leafy greens to be washed before being used in salads, wash water is another potential avenue for acquiring a liquid sample from such products. A purchased salad mix containing romaine lettuce, kale, spinach, and other greens was rinsed using a salad spinner, with the wash water being collected. This wash water was subsequently spiked with 103 or 105 CFU mL−1 E. coli. Both of these contamination concentrations were detected by the platform, visually and when quantified (FIG. 18C, 18D).

Up until this point for the experiments with leafy greens, the extracted surface or wash water was being directly spiked with a given concentration of E. coli. For an actual contaminated product, the bacteria may not necessarily all be extracted into the liquid sample under these conditions. To account for this, another test was completed, this time involving contaminating the food product directly before extracting a liquid sample. This time a stomaching approach was used to break down the product, in order to more accurately simulate the process of sample collection (FIG. 18E).

Leafy greens from the previously mentioned salad mix were contaminated with E. coli at concentrations on the order of 105 and 107 CFU g−1. Liquid samples were then extracted using a stomaching approach, as described in the methods. The recovered E. coli concentrations in the liquid samples after stomaching were on the order of 104 and 106 CFU mL−1 as determined by selective plating, with the control displaying no E. coli growth (FIG. 18F). These liquid samples were then diluted before being added to the sensing platform, to prevent interference from the large amount of particulates. Detection was successful at both tested concentrations, shown in the optical images and the graph (FIG. 18G, 18H). It is also notable that during selective plating on MacConkey agar all samples including the control displayed some non-E. coli bacterial growth, as evidenced by the presence of non-lactose fermenting colonies. This is to be expected, as food samples such as these are non-sterile and will contain some microorganisms. The ability of the sensing platform to specifically detect E. coli without false positive signals for other bacteria is critical for its application in food testing, which often involves the presence of multiple non-target bacteria.

### CONCLUSION

In our previous work, we developed a novel, phage-amplified hydrogel biosensing platform for the on-site colourimetric detection of E. coli in water and urine.10 Now in this work we have significantly expanded the potential real-world applications of the platform, by demonstrating its compatibility with a wide variety of foods that have been vectors for E. coli infection. The successful detection of E. coli contamination from food matrices including milk, RTE chicken purge, and leafy greens supports the possible utility of this platform for on-site contamination monitoring along the food supply chain. With this success in mind, a potential avenue of future exploration is to develop similar platforms for the detection of other foodborne pathogens of interest. DNAzymes for other such bacteria including Salmonella Typhimurium exist,18 and adapting the DNAzyme crosslinked hydrogel platform is a possible future research direction. Another possible line of inquiry is methods of incorporating the sensor into food packaging. In-package biosensing to detect foodborne pathogen contamination is an active area of research, offering the benefit of continuous monitoring.19 Overall, this publication combined with our previous work demonstrates the strong versatility of our sensing platform, including its applicability to on-site clinical testing, water, and food safety monitoring. This wide-ranging functionality, as well as its naked-eye colourimetric signal, makes it a promising candidate for future real-world applications in the food production pipeline.

### Experimental Section

Materials: The following reagents were purchased from Millipore Sigma (Ontario, Canada): Ammonium persulfate (APS), N,N,N′,N′-Tetramethyl ethylenediamine (TEMED), 40% acrylamide monomer solution, gold (III) chloride solution, trisodium citrate dihydrate, bovine serum albumin (BSA), and 1.0 M MgCl2 buffer. Bacto™ yeast extract and dehydrated MacConkey agar were purchased from ThermoFisher Scientific (Ontario, Canada). Oligonucleotides with the acrydite modification were custom ordered from Integrated DNA Technologies (IDT) (Iowa, USA). All food products were purchased from local grocery stores in Hamilton (Ontario, Canada).

Bacterial Preparation: E. coli K12 was cultured from a glycerol stock in LB media. Overnight culture was grown in a shaking incubator at 37° C. and 180 RPM. Before use in the experiments, the liquid culture was centrifuged for 15 minutes at 7000 RCF, 4° C., before being resuspended in ultrapure water.

Milk Sample Preparation: Skim milk was spiked with resuspended E. coli K12 to a final concentration of 105 CFU mL−1. This contaminated milk was then serially diluted in ultrapure water to a 1:100 dilution before being added to the sensing platform.

RTE Chicken Sample Preparation: Liquid from the bottom of RTE rotisserie chicken trays was collected. This chicken purge was spiked with resuspended E. coli K12 to a final concentration of 105 CFU mL−1, before being serially diluted in ultrapure water to a 1:100 dilution.

RTE Egg Sample Preparation: Liquid from a package containing a hardboiled RTE egg was collected, and spiked with resuspended E. coli K12 to a final concentration of 105 CFU mL-1. This contaminated liquid was added to the sensor without dilution.

Carrot Sample Preparation: Liquid from a package of RTE baby-cut carrots was collected, and spiked with resuspended E. coli K12 to a final concentration of 105 CFU mL−1. This contaminated liquid was added to the sensor without dilution.

Leafy Green Sample Preparation: For the iceberg lettuce, mist machines at the grocery store resulted in droplets of water on the surface of the head of lettuce. These droplets were collected, and spiked with resuspended E. coli K12 to a final concentration of 105 CFU mL−1. This contaminated liquid was added to the sensor without dilution. For next leafy greens experiment, 142 g of salad mix containing romaine lettuce, kale, spinach, and other greens was rinsed with 250 mL of water in a salad spinner. The resulting wash water was spiked with resuspended E. coli K12 to final concentrations of 103 and 105 CFU mL−1. This contaminated liquid was added to the sensor without dilution. For the final experiment, 1.5 g of salad mix was contaminated with 105 or 107 CFU E. coli K12. 10 mL of ultrapure water was added to each tube of 1.5 g salad mix, and each sample was homogenized using a stomaching approach. The resulting liquid was then serially diluted in ultrapure water to a 1:100 dilution before being added to the sensing platform.

Bacterial Plating: Liquid samples were serially diluted in triplicate and then plated on selective MacConkey agar. Plates were subjected to overnight incubation at 37° C., followed by colony counting to determine CFU mL−1.

Sensor Fabrication: Hydrogel sensors were fabricated using the procedure outlined in our previous publication.10 Briefly, acrylamide monomers were co-polymerized with each of the two acrydite modified oligonucleotides, through APS and TEMED-induced free radical polymerization at 45° C. BSA coated gold nanoparticles were added. The solution co-polymerized with the DNAzyme oligonucleotide was then mixed with the solution co-polymerized with the substrate oligonucleotide over a heat block at 95° C., in small PCR tubes containing 3 uL of each solution. Complementary binding between the oligonucleotides resulted in the formation of gels. The resulting 6 uL gels were kept frozen at −20° C. until ready to be used. 24 hours before an experiment, these gels were taken out of the freezer and pipetted into smaller 2 uL gels, to each of which 15 uL of MgCl2 buffer was added.

Sensor Use: To each tube containing the hydrogel sensor and buffer, 15 uL of 50 mg mL-1 autolyzed yeast extract and 70 uL of the potentially contaminated sample were added. After incubation at 37° C. and 80 RPM for 6 hours in a Thermo Scientific MaxQ 6000 shaking incubator, 70 uL of T7 bacteriophage at a concentration of 109 plaque forming units (PFU) mL-1 (prepared as described in our previous publication) 10 was added to each tube. 12 hours in the shaking incubator later, after a total incubation time of 18 hours, samples were removed for imaging.

Image Quantification: Tubes containing the gel samples were imaged on the Epson Perfection V850 Pro scanner. ImageJ software was used to quantify the mean brightness of a specific, consistently defined area of each tube.

Statistical Analysis: All graphs were created and statistics analyzed in GraphPad Prism. Unpaired two-tailed t-tests with Welch's correction were used to compare two groups. One-way Brown-Forsythe ANOVA with Dunnett's multiple comparison test was used to compare three or more groups. Experiment sample sizes are described in their respective figure captions. Asterisks on the graphs represent significance levels according to ns (p>0.05), * (p≤0.05), ** (p≤0.01), *** (p≤0.001), and **** (p≤0.0001).

### REFERENCES

1. Havelaar, A. H. et al. World Health Organization Global Estimates and Regional Comparisons of the Burden of Foodborne Disease in 2010. PLOS Med. 12, e1001923 (2015).
2. Van Kessel, J. S., Karns, J. S., Gorski, L., McCluskey, B. J. & Perdue, M. L. Prevalence of Salmonellae, Listeria monocytogenes, and Fecal Coliforms in Bulk Tank Milk on US Dairies. J. Dairy Sci. 87, 2822-2830 (2004).
3. Luna-Guevara, J. J., Arenas-Hernandez, M. M. P., Martinez De La Peña, C., Silva, J. L. & Luna-Guevara, M. L. The Role of Pathogenic E. coli in Fresh Vegetables: Behavior, Contamination Factors, and Preventive Measures. Int. J. Microbiol. 2019, 1-10 (2019).
4. Zarei, O., Shokoohizadeh, L., Hossainpour, H. & Alikhani, M. Y. The Prevalence of Shiga Toxin-Producing Escherichia coli and Enteropathogenic Escherichia coli Isolated from Raw Chicken Meat Samples. Int. J. Microbiol. 2021, 1-5 (2021).
5. Martinez-Chávez, L. et al. Quantitative distribution of Salmonella spp. and Escherichia coli on beef carcasses and raw beef at retail establishments. Int. J. Food Microbiol. 210, 149-155 (2015).
6. Law, J. W.-F., Ab Mutalib, N.-S., Chan, K.-G. & Lee, L.-H. Rapid methods for the detection of foodborne bacterial pathogens: principles, applications, advantages and limitations. Front. Microbiol. 5, (2015).
7. Amiri, M., Bezaatpour, A., Jafari, H., Boukherroub, R. & Szunerits, S. Electrochemical Methodologies for the Detection of Pathogens. ACS Sens. 3, 1069-1086 (2018).
8. Nnachi, R. C. et al. Biosensors for rapid detection of bacterial pathogens in water, food and environment. Environ. Int. 166, 107357 (2022).
9. Vaisocherová-Lisalová, H. et al. Low-fouling surface plasmon resonance biosensor for multi-step detection of foodborne bacterial pathogens in complex food samples. Biosens. Bioelectron. 80, 84-90 (2016).
10. Mann, H. et al. Bacteriophage-Activated DNAzyme Hydrogels Combined with Machine Learning Enable Point-of-Use Colorimetric Detection of Escherichia coli. Adv. Mater. 37, 2411173 (2025).
11. Nutiu, R. & Li, Y. Structure-Switching Signaling Aptamers. J. Am. Chem. Soc. 125, 4771-4778 (2003).
12. Yousefi, H., Ali, M. M., Su, H.-M., Filipe, C. D. M. & Didar, T. F. Sentinel Wraps: Real-Time Monitoring of Food Contamination by Printing DNAzyme Probes on Food Packaging. ACS Nano 12, 3287-3294 (2018).
13. Solomon, E. B., Pang, H.-J. & Matthews, K. R. Persistence of Escherichia coli O157:H7 on Lettuce Plants following Spray Irrigation with Contaminated Water. J. Food Prot. 66, 2198-2202 (2003).
14. Machado-Moreira, B., Richards, K., Brennan, F., Abram, F. & Burgess, C. M. Microbial Contamination of Fresh Produce: What, Where, and How? Compr. Rev. Food Sci. Food Saf 18, 1727-1750 (2019).
15. Mor-Mur, M. & Yuste, J. Emerging Bacterial Pathogens in Meat and Poultry: An Overview. Food Bioprocess Technol. 3, 24-35 (2010).
16. U.S. FDA. Outbreak Investigation of E. coli O121:H19: Organic Carrots (November 2024). (2024).
17. Kintz, E. et al. Outbreaks of Shiga Toxin-Producing Escherichia coli Linked to Sprouted Seeds, Salad, and Leafy Greens: A Systematic Review. J. Food Prot. 82, 1950-1958 (2019).
18. Prasad, A. et al. Advancing In Situ Food Monitoring through a Smart Lab-in-a-Package System Demonstrated by the Detection of Salmonella in Whole Chicken. Adv. Mater. 35, 2302641 (2023).
19. Khan, S. et al. Material Breakthroughs in Smart Food Monitoring: Intelligent Packaging and On-Site Testing Technologies for Spoilage and Contamination Detection. Adv. Mater. 36, 2300875 (2024).

While the present disclosure has been described with reference to examples, it is to be understood that the scope of the claims should not be limited by the embodiments set forth in the examples, but should be given the broadest interpretation consistent with the description as a whole.

All publications, patents and patent applications are herein incorporated by reference in their entirety to the same extent as if each individual publication, patent or patent application was specifically and individually indicated to be incorporated by reference in its entirety. Where a term in the present disclosure is found to be defined differently in a document incorporated herein by reference, the definition provided herein is to serve as the definition for the term.

1. A biosensor for detecting the presence of a target molecule in a sample, the biosensor comprising:
a polymeric hydrogel;
a colorimetric indicator entrapped in the polymeric hydrogel; and
an oligonucleotide that crosslinks the polymeric hydrogel, wherein the oligonucleotide is separated in the presence of the target molecule, thereby releasing the colorimetric indicator from the hydrogel.

2. The biosensor of claim 1, wherein the target molecule is a protein from a bacterial target.

3. The biosensor of claim 1, wherein the bacterial target comprises E. coli.

4. The biosensor of claim 2, further comprising a lytic bacteriophage that is capable of infecting and lysing the bacterial target, thereby increasing the concentration of the protein in the sample.

5. The biosensor of claim 4, wherein the bacteriophage comprises Bacteriophage T7.

6. The biosensor of claim 1, wherein the colorimetric indicator comprises gold nanoparticles.

7. The biosensor of claim 1, wherein the colorimetric indicator is bulked with a BSA coating.

8. The biosensor of claim 1, wherein the polymeric hydrogel comprises acrylamide.

9. The biosensor of claim 1, wherein the oligonucleotide is acrydite-modified.

10. The biosensor of claim 1, wherein the oligonucleotide comprises a substrate strand hybridized to a DNAzyme strand, wherein the DNAzyme strand cleaves the substrate strand in the presence of the protein specific to the target.

11. The biosensor of claim 10, wherein the DNAzyme strand comprises the sequence GA TGT GCG TCT TGA TCG AGA CCT GCG ACA GGA AG (SEQ ID NO: 1).

12. The biosensor of claim 10, wherein the substrate strand comprises the sequence TT TTT ACT CTT CCT AGC TrAT GGT TCG ATC AAG A (SEQ ID NO: 2).

13. The biosensor of claim 1, wherein the biosensor has a sensitivity of greater than about 10¹ CFU mL⁻¹.

14. The biosensor of claim 1, wherein the biosensor produces a visible result in less than about 18 hours.

15. The biosensor of claim 1, wherein no equipment is needed for analysis of the test result.

16. The biosensor of claim 1, wherein the biosensor is stable for at least two weeks at temperatures between about −20° C. and about 20° C.

17. The biosensor of claim 1, wherein the sample is a liquid sample from water, a bodily fluid, a food source, and/or a surface.

18. A method of detecting a target molecule in sample, the method comprising applying the sample to the biosensor of claim 1.

19. The method of claim 18, further comprising use of an AI network to assess the colorimetric result.

20. A method of amplifying DNAzyme signalling in a biosensor that detects a bacteria-specific protein from whole bacteria, the method comprising applying to the sensor a lytic bacteriophage that infects and lyses the bacteria.
